(19) United States
(12) Patent Application Publication
(10) Pub. No.: US 2023/0257690 A1
(43) Pub. Date: Aug. 17, 2023

(54) CELL CULTURE AUTOFILL SYSTEM

(71) Applicant: CORNING INCORPORATED, Corning, NY (US)

(72) Inventors: Scott Matthew Bennett, Gray, ME (US); Thomas Albert Cloutier, Arundel, ME (US); William Joseph Lacey, North Andover, MA (US); Gregory Roger Martin, Acton, ME (US); Allison Jean Tanner, Portsmouth, NH (US)

(21) Appl. No.: 18/018,418

(22) PCT Filed: Jul. 28, 2021
(86) PCT No.: PCT/US2021/043383

§ 371 Date: Jan. 27, 2023

Related U.S. Application Data
(60) Provisional application No. 63/058,796, filed on Jul. 30, 2020.

(51) Int. Cl. C12M 1/00; C12M 1/34; C12M 1/12; C12M 3/00

(52) U.S. Cl. CPC C12M 23/50; C12M 41/44; C12M 25/06; C12M 23/48; C12M 29/06

(57) ABSTRACT

A cell culture system for culturing cells is provided. The cell culture system including a cell culture vessel with a cell culture chamber for culturing cells, an inlet through which liquid can flow for filling the cell culture vessel, and an outlet through which fluid can exit the cell culture vessel. The system also includes at least one fill sensor arranged to detect liquid within the cell culture vessel reaching a fill level at a predetermined position of the cell culture vessel during filling of the cell culture vessel, where the at least one fill sensor can generate a detection signal when liquid within the cell culture vessel reaches the fill level. Also included is an actuator to change an orientation of the cell culture vessel in response to the detection signal.

### CROSS-REFERENCE TO RELATED APPLICATIONS

This is a national stage application under 35 U.S.C. § 371 of International Application No. PCT/US2021/043383, filed Jul. 28, 2021, which claims the benefit of priority under 35 U.S.C. § 119 of U.S. Provisional Application Ser. No. 63/058,796 filed on Jul. 30, 2020, the content of which is relied upon and incorporated herein by reference in its entirety.

### FIELD

The present disclosure relates to multi-position supports for cell culture apparatuses and, in particular, multi-position supports having both an upright configuration and a tilted configuration.

### BACKGROUND

Many types of cell culture articles are constructed to provide stacked or stackable units for culturing cells. For example, T-flasks are typically made to have flat top and bottom surfaces that allow T-flasks to be stacked, providing space savings. Some modified T-flasks have multiple parallel culture surfaces within the flask to reduce time and effort associated with filling and emptying. Other culture apparatuses are multi-component assemblies having a plurality of parallel or stacked culture surfaces. With most of such stacked culture assemblies, each culture layer is isolated to reduce hydrostatic pressure on the lower culture layers. As the number of stacked layers increases, the potential effect of hydrostatic pressure increases.

One exemplary cell culture article is Corning's HYPERStack™ system. The HYPERStack™ system includes multiple modules formed of individual stackette layers that can be interconnected by flexible tubes that connect to tube connectors. The modules are interconnected for filling and emptying the HYPERStack™ system. Valves and other devices may be used to control fluid flow into and out of the HYPERStack™ system. The use of these valves and other devices can be cumbersome and provide potential leak locations.

Current processes for filling and emptying the HYPERStack™ system are inconsistent because the filling and emptying protocols involve tilting the HYPERStack™ system at various stages in order to yield better results. Without an accessory to drive this protocol, users have resorted to using whatever is on hand in the lab, such as tubing clamps, tube racks, doorstops, etc. What is needed is a multi-position support that can be used to handle cell culture apparatuses and reliably place them at multiple tilted angles during fill and empty procedures.

Even with improved accessories or a multi-position support, manual filling and emptying requires careful attention be paid by the user. For example, it is possible to fill units too fast or with too much media, which can cause undue stress on the vessel and/or can occlude an air vent filter. These problems can cause leaks or contamination of the vessels during use. These challenges are amplified when a user is attempting to fill or use multiple cell culture devices at once. To avoid these problems, a semi-automated or fully-automated cell culture system is needed that can monitor the units while they are being filled with media.

### BRIEF SUMMARY

According to embodiments of this disclosure, a cell culture system is provided. The cell culture system includes a cell culture vessel with a cell culture chamber for culturing cells, an inlet through which liquid can flow for filling the cell culture vessel, and an outlet through which fluid can exit the cell culture vessel. The system also includes at least one fill sensor arranged to detect liquid within the cell culture vessel reaching a fill level at a predetermined position of the cell culture vessel during filling of the cell culture vessel. The at least one fill sensor can generate a detection signal when liquid within the cell culture vessel reaches the fill level. The system further includes an actuator to change an orientation of the cell culture vessel in response to the detection signal.

In further aspects of embodiments of this disclosure, the at least one fill sensor can include a first fill sensor arranged to detect liquid within the cell culture vessel reaching a first fill level and can generate a first detection signal when liquid within the cell culture vessel reaches the first fill level. The actuator can then change the orientation of the cell culture vessel from a first orientation to a second orientation in response to the first detection signal.

The at least one fill sensor can also include a second fill sensor arranged to detect liquid within the cell culture vessel reaching a second fill level and can generate a second detection signal when liquid within the cell culture vessel reaches the second fill level. The system can then stop filling the cell culture vessel in response to the second detection signal. The second fill level is different than the first fill level.

As an aspect of some embodiments, the cell culture vessel is a multi-layered cell culture vessel.

As an additional aspect, the system can further include a controller to receive the detection signal of the at least one sensor and to control the actuator in response to the detection signal. The system can further include a pump fluidly connected to the inlet of the cell culture vessel. The controller can control a flow rate of the pump.

According to aspects of some embodiments, the cell culture includes a fill side and a support-facing side. The fill side is where the inlet and the outlet are disposed, and the support-facing side is adjacent to the fill side and faces a substantially horizontal support member that is underneath the cell culture vessel in the first orientation. In the first orientation, the support-facing side is at a first angle relative to the support member, and the outlet is at a first distance from the support member, the inlet being closer to the support member than the outlet. In the second orientation, the support-facing side is at a compound angle relative to the support member, the compound angle comprising (i) a second angle between a length of the support-facing side and the support member and (ii) a third angle between a width of the support-facing side and the support member. Also, in the second orientation, the outlet is at a second distance from the support member, the second distance being greater than the first distance.

According to aspects of some embodiments, the cell culture vessel may include multiple cell culture chambers, also referred to as stacks or layers, each chamber stacked one on top of another, wherein a bottom surface of each cell culture chamber comprises a cell culture surface. The cell culture vessel may comprise a top-most surface, a bottom-most surface, and four sides extending between the top-most surface and bottom-most surface. A first side comprises a portion of the inlet access column or fill column and a portion of the outlet access column or vent column; a third side is opposite the first side; a second side is adjacent the first side and third side and comprises a portion of the inlet access column or fill column along an end adjacent the first side; a fourth side is opposite the second side and adjacent the first side and third side and comprises a portion of the outlet access column or vent column along an end adjacent the first side. An inlet port or fill port and an outlet port or vent port may be disposed on the top-most surface of the vessel along one side of the vessel and arranged in opposite corners or at opposite ends of that side of the vessel. The cell culture vessel may further comprise an inlet access column or fill column in communication with each chamber and with the inlet port or fill port. The cell culture vessel may further comprise an outlet access column or vent column in communication with each chamber and with the outlet port or vent port. The inlet access column or fill column extends vertically from the inlet port or fill port on a top of the vessel to a bottom-most chamber in the vessel and may be disposed in a corner of a vessel. The outlet access column or vent column extends vertically from the outlet port or vent port on a top of the vessel to a bottom-most chamber in the vessel. The inlet access column or fill column may be disposed in a corner of a vessel, and the outlet access column or vent column may be disposed on a same side of the vessel but in a corner of the vessel opposite from the inlet access column or fill column. In a first orientation, the cell culture vessel is in an incubation orientation with the top-most surface comprising the inlet and outlet ports. In a first fill orientation, the cell culture vessel is rotated 90 degrees from the first orientation so that the outlet port is positioned above the inlet port in a vertical direction and the top-most surface faces outward instead of upward. In a second fill orientation, the cell culture vessel is rotated 90 degrees from the first fill orientation so that a side opposite the inlet and outlet column is horizontal to the support surface. outlet port and the inlet port are horizontal to one another away from the support surface, with the top-most surface facing outward.

In further aspects of some embodiments, the at least one fill sensor is attached to an exterior of the cell culture vessel and detects the fill level through a wall of the cell culture vessel. The cell culture vessel can include multiple cell culture chambers and a manifold connecting the multiple cell culture chambers, while the at least one fill sensor can be attached to the manifold. The cell culture vessel can include multiple cell culture chambers and an inlet access column or fill column connecting the multiple cell culture chambers, while the at least one fill sensor can be attached to the inlet access column. In some embodiments, the at least one fill sensor is detachable from the cell culture vessel and is reusable. The at least one fill sensor can be at least one of an optical sensor, a through-beam sensor, or a photoelectric sensor.

As a further aspect of embodiments, the system includes a flow control arranged to detect in-line fluid pressure of the system. The flow control can cause a reduction of a flow rate of the system in response to the in-line fluid pressure being equal to or greater than a predetermined pressure value.

In additional aspects, the system further includes a valve at an outlet of the cell culture vessel. The valve can close a fluid path at or near the outlet of the cell culture vessel to stop filling of the cell culture vessel. The valve can be a pinch valve on an exterior of tubing connected to the outlet. The valve can be designed to close the fluid path in response to at least one of the detection signal from one of the at least one fill sensors and an in-line fluid pressure.

In further aspects of some embodiments, the system further includes a multi-position support to support the cell culture vessel. The actuator can be attached to the multi-position support and can change an orientation of the multi-position support relative to a horizontal support surface upon which the multi-position support is disposed.

In additional aspects of some embodiments, the actuator has an adjustable length including a first length and a second length. At the first length the cell culture vessel is in the first orientation and at the second length the cell culture vessel is in the second orientation. The first length can be greater than the second length. The actuator can include at least one of a solenoid, a piezoelectric material, a pneumatic piston, and a hydraulic piston.

Additional aspects of some embodiments include the system further including a third fill sensor arranged to detect liquid within the cell culture reaching a third fill level. The third fill sensor can generate a third detection signal when liquid within the cell culture vessel reaches the third fill level, the third fill level being different than the first fill level and the second fill level. The controller can be configured to slow a filling rate of the cell culture vessel based on a third detection signal from the third fill sensor.

As an aspect of some embodiments, during filling of the cell culture vessel with a liquid, the liquid reaches the first fill level before the second fill level. In some embodiments, during filling of the cell culture vessel with a liquid, the liquid reaches the third fill level after the first fill level and before the second fill level.

As an aspect of embodiments, the controller can to stop filling the cell culture vessel based on the second detection signal from the second fill sensor.

In further aspects of some embodiments, the multi-position support includes a primary base that rests against the support member in an upright configuration; a support surface that is offset vertically from the primary base in the upright configuration, the support surface supporting the cell culture vessel with the cell culture vessel located thereon; and an intermediate surface that extends between the primary base and the support surface, wherein the intermediate surface meets the primary base at an interface that extends at an oblique angle to sides of the primary base. The multi-position support has a tilted configuration where the multi-position support is rotated about the interface such that the support surface is closer to the support member than in the upright configuration with the support surface supporting the cell culture vessel thereon.

The multi-position support can further include a secondary base that rests against the support member in the upright configuration. In some embodiments, the support surface is a first support surface, and the multi-position support further includes a second support surface located between the primary base and the secondary base, the second support surface supporting the cell culture vessel with the cell culture vessel located thereon. The first support surface and the second support surface can lie in substantially a same plane that is oblique to the primary base. The multi-position support can further include a support flange that engages a fill side of the cell culture vessel to constrain the cell culture vessel on the first and second support surfaces. The multi-position support can further include another support flange that extends outward from the first support surface that engages a rear side of the cell culture vessel that is opposite the fill side. The second support surface can be spaced from the support member. In aspects of some embodiments, the support surface meets the intermediate surface at another interface that is at an oblique angle to sides of the primary base. The oblique angles of both interfaces to the sides of the primary base can be about the same.

According to additional embodiments, a method is provided of changing a fill angle of a cell culture apparatus including cell culture vessel with multiple cell culture modules, the multiple cell culture modules being fluidly connected together by a fluid manifold and an air manifold. The method includes: connecting the cell culture apparatus to a multi-position support, the multi-position support including a tilted configuration and an upright configuration; filling the cell culture apparatus while supported by the multi-position support with the multi-position support in either the upright configuration or the tilted configuration; detecting with a first fill sensor exterior to the cell culture modules when the cell culture apparatus has been filled to a first fill level; changing the multi-position support from one of the tilted configuration and the upright configuration to the other of the tilted configuration and the upright configuration, based on the detecting by the first fill sensor; detecting with a second fill sensor exterior to the cell culture modules when the cell culture apparatus has been filled to a second fill level different from the first fill level; and stopping the filling based on the detecting by the second fill sensor.

In aspects of some embodiments of the method, the multiple cell culture modules each contain multiple layers of cell culture chambers.

In further aspects of some embodiments of the method, the multi-position support can include: a primary base that rests against a support member in the upright configuration; a support surface that is offset vertically from the primary base in the upright configuration, the support surface supporting the multi-layer cell culture apparatus with the multi-layer cell culture apparatus located thereon; and an intermediate surface that extends between the primary base and the support surface, wherein the intermediate surface meets the primary base at an interface that extends at an oblique angle to sides of the primary base. In the tilted configuration, the multi-position support is rotated about the interface such that the support surface is closer to the support member than in the upright configuration with the support surface supporting the multi-layer cell culture apparatus thereon.

In aspects of some embodiments of the method, the step of filling includes filling the cell culture apparatus while supported by the multi-position support with the multi-position support in the upright configuration. The method can further include tilting the cell culture apparatus using the multi-position support by rotating the multi-position support about the interface. In further aspects of the method, the changing of the multi-position support from one of the tilted configuration and the upright configuration to the other includes moving an actuator attached to the multi-position support.

Described herein are cell culture filling systems that semi- or fully-automate filling and emptying of a cell culture system by automatically controlling the orientation and/or flow rates of the cell culture vessel during filling or emptying of the vessel with liquid media, the orientation being switchable between upright and tilted configurations that place the cell culture vessel in different angular orientations relative to horizontal. By providing the cell culture vessel with different angular orientations, improved fill and empty results can be achieved in a more reliable, consistent, and efficient fashion. Further, in the tilt configuration, the fill side (front) of the cell culture vessel is provided with a compound angle up where an air manifold is elevated in both top-to-bottom and front-to-rear directions.

### BRIEF DESCRIPTION OF THE DRAWINGS

FIG. 1 is a perspective view of a cell culture apparatus including manifolds, according to one or more embodiments shown and described herein.

FIG. 2 is a diagrammatic of multiple stackette layers for use with the cell culture apparatus of FIG. 1, according to one or more embodiments shown and described herein.

FIG. 3 is a side view of a multi-position support that supports the cell culture apparatus of FIG. 1 in an upright configuration, according to one or more embodiments shown and described herein.

FIG. 4 is a perspective view of the multi-position support of FIG. 3, according to one or more embodiments shown and described herein.

FIG. 5 is a plan view of the multi-position support of FIG. 4, according to one or more embodiments shown and described herein.

FIG. 6 is a side view of the multi-position support of FIG. 3 in a tilted configuration, according to one or more embodiments shown and described herein.

FIG. 7 is an end view of the multi-position support of FIG. 6 in the tilted configuration, according to one or more embodiments shown and described herein.

FIG. 8 is a perspective view of a cell culture autofill system in a first orientation, according to one or more embodiments shown and described herein.

FIG. 9 is a perspective view of a cell culture autofill system of FIG. 8 in a second orientation.

FIG. 10 is a perspective view of a cell culture apparatus, according to one or more embodiments shown and described herein.

FIG. 11 is a side view of the cell culture apparatus of FIG. 10 in a first fill orientation, according to one or more embodiments shown and described herein.

FIG. 12 is a side view of the cell culture apparatus of FIG. 10 in a second fill orientation, according to one or more embodiments shown and described herein.

FIG. 13 is a side view of the cell culture apparatus of FIG. 10 in an incubation orientation, according to one or more embodiments shown and described herein.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.”

The present disclosure describes cell culture systems that enable automated filling and/or emptying of cell culture vessels with liquid media. It is contemplated that embodiments of the system can include different combinations of various components discussed herein, including one or more of a cell culture vessel; a fill sensor for detecting a fill level of the cell culture vessel; an actuator for changing an orientation of the cell culture vessel during filling; a multi-position support for supporting the cell culture vessel; a controller; a pressure sensor; and various connections, fittings, tubing, and manifolds. Embodiments described herein use fill sensors for monitoring the level of liquid media during filling or emptying of the vessel and reorient the cell culture vessel or adjust the filling rate depending on the fill level. The systems and methods disclosed herein can enable semi-automated or fully-automated filling and/or emptying of a cell culture vessel. As a result, cell culture systems and methods are provided that lower the risk of leaks, contamination, and other stresses on the cell culture system, and the degree of user monitoring and attention needed during the filling or emptying procedure is decreased.

As described above, the present disclosure describes multi-position supports for multi-layer cell culture apparatus. The multi-position supports may be formed by bending a plate to include a primary base that rests against a support member in an upright configuration. A support surface is provided that is offset vertically from the primary base in the upright configuration and supports a multi-layer cell culture apparatus at an angle to the support surface, or at an angle to horizontal. The multi-position supports further include an intermediate surface that extends between the primary base and the support surface. The intermediate surface meets the primary base at an interface that extends at an oblique angle to sides of the primary base. The multi-position support has a tilted configuration where the multi-position support is rotated about the interface on the support surface such that the support surface is closer to the support member than in the upright configuration with the support surface supporting the multi-layer cell culture apparatus thereon.

The multi-layer cell culture apparatus includes cell culture modules that include a plurality of growth or culture surfaces in cell culture chambers coupled together via manifolds to form the cell culture devices. The cell culture modules can be further coupled to additional cell culture modules via manifolds to form stacked cell culture devices. The plurality of culture surfaces may be stacked in a multi-layer configuration. The manifold can include an integral column structure that is formed as a monolithic part of the manifold. The column structure includes an inlet port and provides at least part of a fluid flow pathway from the inlet port that is in fluid communication with the individual cell culture chambers within the cell culture modules. The manifolds and associated column structures may provide a closed system where the column structures can be connected to flexible tubing to isolate the cell culture chambers from the environment during use of the cell culture apparatuses.

In embodiments of this disclosure, one or more sensors can be used to measure a fill level in the cell culture vessel or the manifold. The filling rate across cell culture devices can vary somewhat, so if a user is attempting to fill multiple vessels at once, the sensor on each cell culture apparatus can determine the appropriate time for each specific vessel to be re-oriented or for fluid flow to be changed.

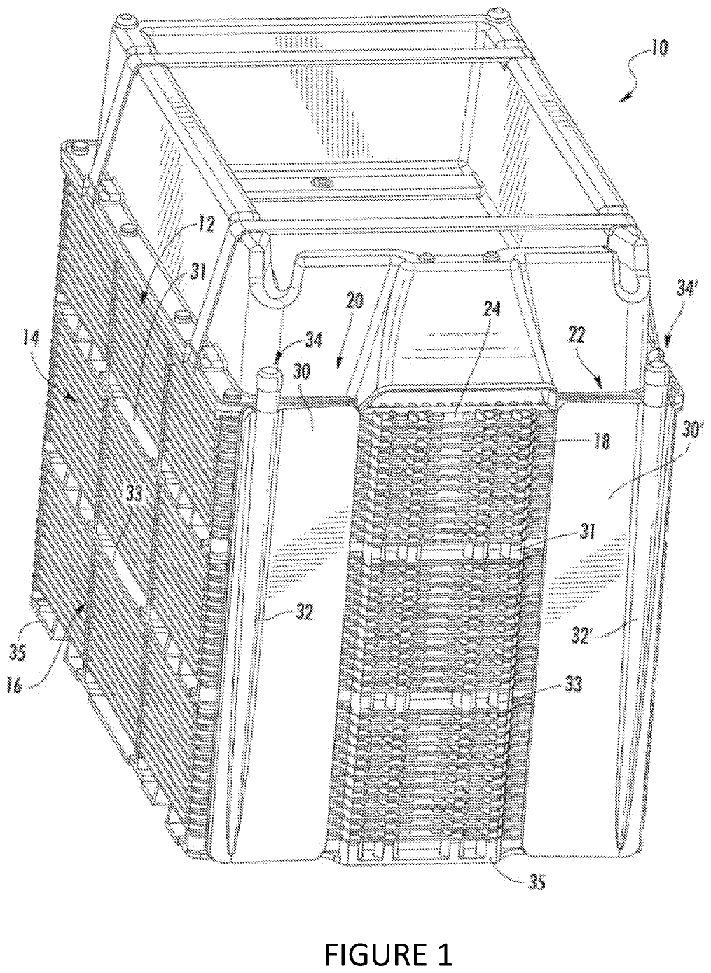

Referring to FIG. 1, a cell culture apparatus 10 includes three cell culture modules 12, 14, and 16, each containing multiple layers of cell culture chambers 18, and are stacked, one on top of the other, to form the multiple layer cell culture apparatus 10. Each cell culture module 12, 14, and 16 utilizes two manifolds 20 and 22. Liquid may enter and exit the cell culture modules 12, 14, and 16 through the first manifold 20. Thus, the first manifold 20 may be referred to as a fluid manifold. Air may enter and exit the cell culture modules 12, 14, and 16 through the second manifold 22. Thus, the second manifold 22 may be referred to as an air manifold.

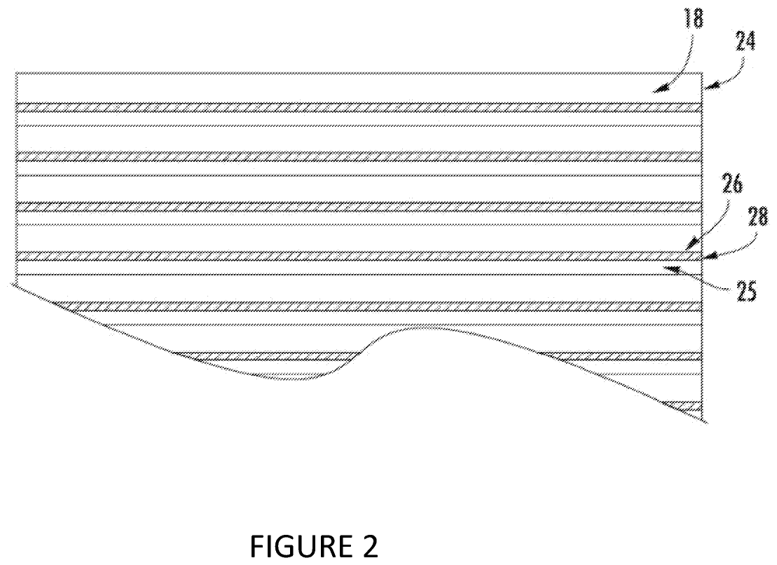

The cell culture modules 12, 14, and 16 may each include multiple stackette layers 24 that, when stacked together, form the multiple cell culture chambers 18 having tracheal spaces (air spaces) 25 there between, as shown in FIG. 2. FIG. 2 is a schematic representation of the multiple stackette layers 24 that are stacked together to form the layered cell culture chambers 18 and cell culture surfaces 26 that include a gas permeable, liquid impermeable film 28, for example, the stackette layers 24 include the tracheal spaces 25 to allow transfer of gasses between the cell culture chambers 18 and the exterior of the cell culture apparatus 10. Referring back to FIG. 1, the cell culture modules 12, 14, and 16 may be separated from one another by spacers 31, 33, and 35. The spacers 31, 33, and 35 can provide structural support for the individual cell culture modules 12, 14, and 16. In some embodiments, spacers 31 and/or 33 may be replaced by additional stackette layers 24 to provide a higher total number of cell culture chambers 18. Further, a riser volume may be provided above the cell culture module 12 to catch residual air, rather than air residing in the cell culture chambers 18.

A cell culture module, or portions thereof, as described herein may be formed from any suitable material. Preferably, materials intended to contact cells or culture media are compatible with the cells and the media. Typically, cell culture modules are formed from polymeric material. Examples of suitable polymeric materials include polystyrene, polymethylmethacrylate, polyvinyl chloride, polycarbonate, polysulfone, polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers, and the like.

In some embodiments, the culture modules contain the gas permeable, liquid impermeable film 28 to allow transfer of gasses between the cell culture chamber 18 and ultimately with the exterior of the cell culture assembly. Such culture modules can include spacers or spacer layers positioned adjacent the film, exterior to the chamber, to allow air flow between stacked units. One commercially available example of a cell culture apparatus containing such stacked gas permeable culture units is Corning's HYPERStack™ cell culture apparatus. Examples of suitable gas permeable polymeric materials useful for forming a film include polystyrene, polyethylene, polycarbonate, polyolefin, ethylene vinyl acetate, polymethylpentene, polypropylene, polytetrafluoroethylene (PTFE), or compatible fluoropolymer, a silicone rubber or copolymer, poly(styrene-butadiene-styrene) or combinations of these materials. As manufacturing and compatibility for the growth of cells permits, various polymeric materials may be utilized. Preferably the film is of a thickness that allows for efficient transfer of gas across the film. For example, a polystyrene film may be of a thickness of about 0.003 inches (about 75 micrometers), though various thicknesses are also permissive of cell growth. As such, the film may be of any thickness, preferably between about 25 and 250 micrometers, or between approximately 25 and 125 micrometers. The film allows for the free exchange of gases between the chamber of the assembly and the external environment and may take any size or shape. Preferably, the film is durable for manufacture, handling, and manipulation of the apparatus.

As mentioned above, the cell culture modules 12, 14, and 16 may be connected together using the manifolds 20 and 22. The manifold 20 includes a side wall base structure 30 and a column structure 32 that is formed as a monolithic part of the side wall base structure 30 providing a unitary manifold 20. The column structure 32 includes a barb structure 34 and provides at least part of a fluid flow pathway from the barb structure 34 that is in fluid communication with the individual cell culture chambers 18 within the cell culture modules 12, 14, and 16. The manifold 20 may be configured to allow filling and emptying of the cell culture chambers 18.

The manifold 22 also includes a side wall base structure 30′ and a column structure 32′ that is formed as a monolithic part of the side wall base structure 30′ providing a unitary manifold 22. The column structure 32′ includes a barb structure 34′ and provides at least part of a fluid flow pathway from the individual cell culture chambers 18 within the cell culture modules 12, 14, and 16 to the barb structure 34′. The manifold 22 may be configured to allow filling and emptying of the cell culture chambers 18 by allowing air to enter and exit the cell culture apparatus 10. In some embodiments, the column structure 32′ may be offset from the illustrated location in order to control media flow into the column structure 32′.

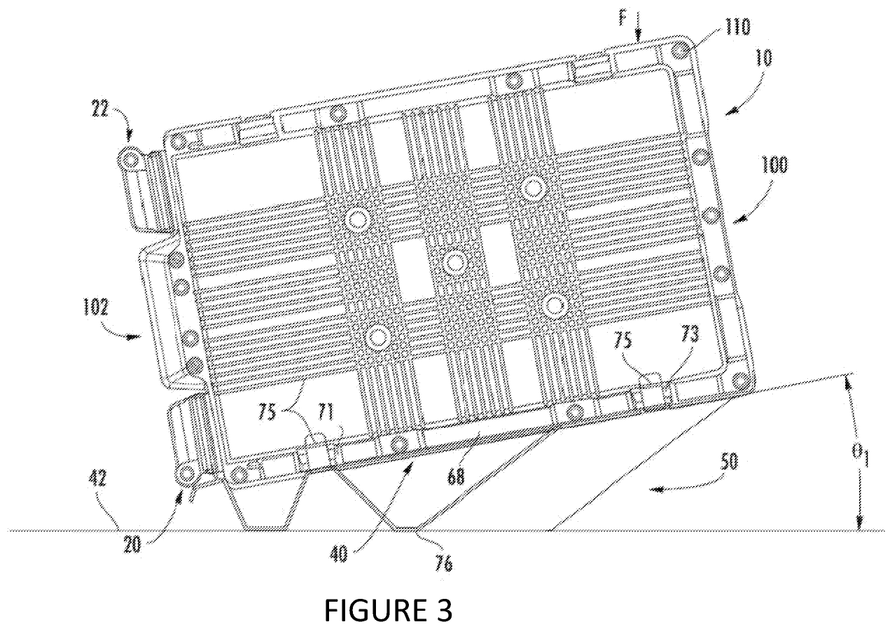

For a typical filling procedure, a cell culture apparatus 10 can be placed with its left side down, facing a support surface or tray. In this orientation, the front of the cell culture apparatus 10 with manifolds 20 and 22 is tilted downward for a first filling orientation at the start of filling (see the side view of FIG. 3). The flow of liquid media into the cell culture vessel is then initiated. For example, the media can be pumped into the lower column structure 32 through the barb structure 34 with the use of a peristatic pump, or the vessel can be filled with gravity induced flow. As the liquid media within the cell culture apparatus 10 and rises to a first fill level at a predetermined position, the cell culture apparatus 10 (and filling tray, if used) is re-oriented to a second filling position. In this second filling orientation, filling can continue until the liquid media reaches a final fill level in the cell culture apparatus 10. Upon reaching the final fill level, the flow of media is stopped and the inlet and outlet from the manifolds 20, 22 can be shut or clamped off to close the system. At this time, the cell culture apparatus 10 is ready for cell culture use.

Embodiments of this disclosure include a type of filling tray or multi-position support, as mentioned above. Details of the multi-position support are presented below to better understand the nature of the filling and/or emptying process of the cell culture apparatus 10, including the nature of the first and second filling orientations of the cell culture apparatus 10 and how the cell culture apparatus 10 is moved from one orientation to another.

Referring to FIG. 3, using the multi-position support 50, the cell culture apparatus 10 may be filled and emptied with the cell culture apparatus lying on a side 40 and tilted, as illustrated by FIG. 3. The cell culture apparatus 10 may be reliably positioned on the side 40 at a predetermined tilt angle Θ₁ (e.g., between about 10 degrees and about 12 degrees) relative to a support member 42 or horizontal using a multi-position support 50. The side 40 closest to the fluid manifold 20 is placed on the multi-position support 50 such that the fluid manifold 20 is lower than the air manifold 22. As will be described in greater detail below, the multi-position support 50 can be tilted between an upright configuration (as shown by FIG. 3) and a tilted configuration to position the cell culture apparatus 10 at different angles relative to horizontal.

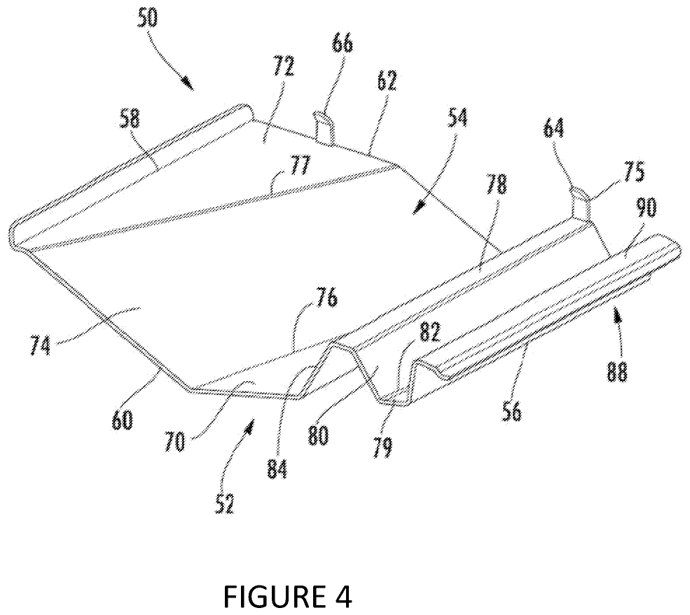

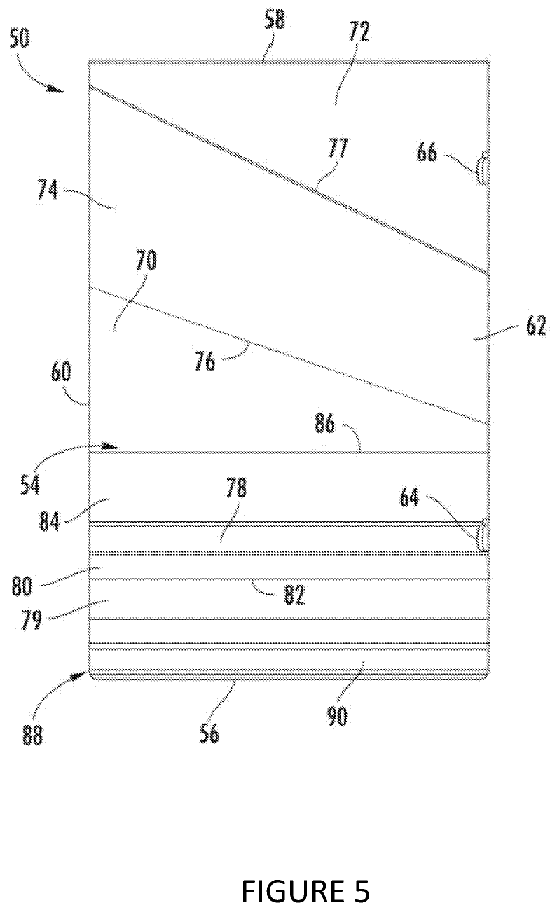

Referring to FIGS. 4 and 5, the multi-position support 50 is illustrated in isolation and is formed as a monolithic, bent plate that includes a bottom 52, a top 54, opposite ends 56 and 58 and opposite sides 60 and 62. At side 62, the multi-position support 50 includes location tabs 64 and 66 that engage a bottom edge 68 of the cell culture apparatus 10 (FIG. 3) with the multi-position support 50 in an upright, standing position and helps to hold the cell culture apparatus 10 in place on the multi-position support 50. In some embodiments, the bottom edge 68 of the cell culture apparatus 10 may be provided with recessed features 71 and 73 that are sized and located to receive the location tabs 64 and 66. The location tabs 64 and 66 may include a bend 75 that can be used to grip the bottom edge 68 and inhibit side-to-side movement of the cell culture apparatus 10 off of the multi-position support 50.

The multi-position support 50 includes a primary base 70 that rests against a support member (e.g., a table or lab bench) with the multi-position support 50 in an upright configuration as shown. A primary support surface 72 is provided that is offset vertically from the primary base 70 in the upright configuration and supports the cell culture apparatus 10 thereon. The multi-position support 50 further includes an intermediate surface 74 that extends between the primary base 70 and the primary support surface 72. The intermediate surface 74 meets the primary base 70 at an interface 76 formed as a bend that extends at an oblique angle to the sides 60 and 62 of the multi-position support 50. The intermediate surface 74 also meets the primary support surface 72 at an interface 77 formed as a bend that extends at an oblique angle to the sides 60 and 62. In some embodiments, the oblique angles of the interfaces 76 and 77 are about the same (e.g., within five degrees) relative to the sides 60 and 62 or they may be different.

The multi-position support 50 further includes a secondary base 79 that rests against the support member with the multi-position support 50 in the upright configuration. A secondary support surface 78 is provided that is offset vertically from the secondary base 79 in the upright configuration and supports the cell culture apparatus 10 thereon. The secondary support surface 78 and the primary support surface 72 lie in a same plane that is at an angle to horizontal and is also oblique to the primary base 70 and the secondary base 79. The multi-position support 50 further includes another intermediate surface 80 that extends between the secondary base 79 and the secondary support surface 78. The intermediate surface 80 meets the secondary base 79 at an interface 82 formed as a bend that extends perpendicular to the sides 60 and 62 of the multi-position support 50. Another intermediate surface 84 extends between the primary base 70 and the secondary support surface 78. The intermediate surface 84 meets the primary base 70 at an interface 86 formed as a bend that also extends perpendicular to the sides 60 and 62. A handle feature 88 is provided at the end 56. The handle feature 88 can also include a support flange 90 that is offset vertically from the secondary base 79 in the upright configuration and supports the cell culture apparatus 10 thereon. The end 58 is provided with a support flange 94 that extends vertically outward from the primary support surface 72 and is used to hold the cell culture device 10 on the primary support surface 72.

FIG. 3 illustrates the multi-position support 50 with the cell culture apparatus 10 supported thereon in the upright configuration. In the upright configuration, the cell culture apparatus 10 has a rear 100 that is more elevated than a front 102 at the angle θ₁ (between 10 degrees and 12 degrees) to horizontal. However, the top to bottom angle is parallel (zero degrees) to horizontal. This upright configuration may place the cell culture apparatus 10 in an initial fill position to begin filling the cell culture apparatus 10 where the front 102 is lower than the rear 100 thereby providing a gentler fill angle, which can reduce foaming in the fluid and promote air evacuation through the air manifold and filter connected thereto.

As the cell culture apparatus 10 is being filled with the multi-position support 50 in the upright configuration, the fluid level within the cell culture apparatus 10 rises toward the air manifold 22 and toward the filter that is connected to the air manifold. Wetting of the filter can reduce airflow rate out of the cell culture apparatus 10 thereby pressurizing the interior, which can lead to an undesirable environment within the cell culture apparatus 10. To reduce a likelihood that fluid reaches the filter, the multi-position support 50 is provided with a tilted configuration where the multi-position support 50 along with the cell culture apparatus 10 is rotated without lifting either the multi-position support 50 or the cell culture apparatus 10. The multi-position support 50 along with the cell culture apparatus 10 can simply be manually tilted by applying a force F to a rear corner 110 of the cell culture apparatus 10, which causes the multi-position support 50 and the cell culture apparatus 10 to rotate about the interface 76. Because the interface 76 extends at the oblique angle to the sides 60 and 62 of the multi-position support 50, the tilting changes both the front to rear angle and the top to bottom angle to increase the elevation of a top of the air manifold where the filter is connected. According to embodiments described below, this tilting operation can also be performed by an automated cell culture system, without the manual application of the force F. However, the same multi-position support 50 shown and described can be used for both the manual and automated tilting.

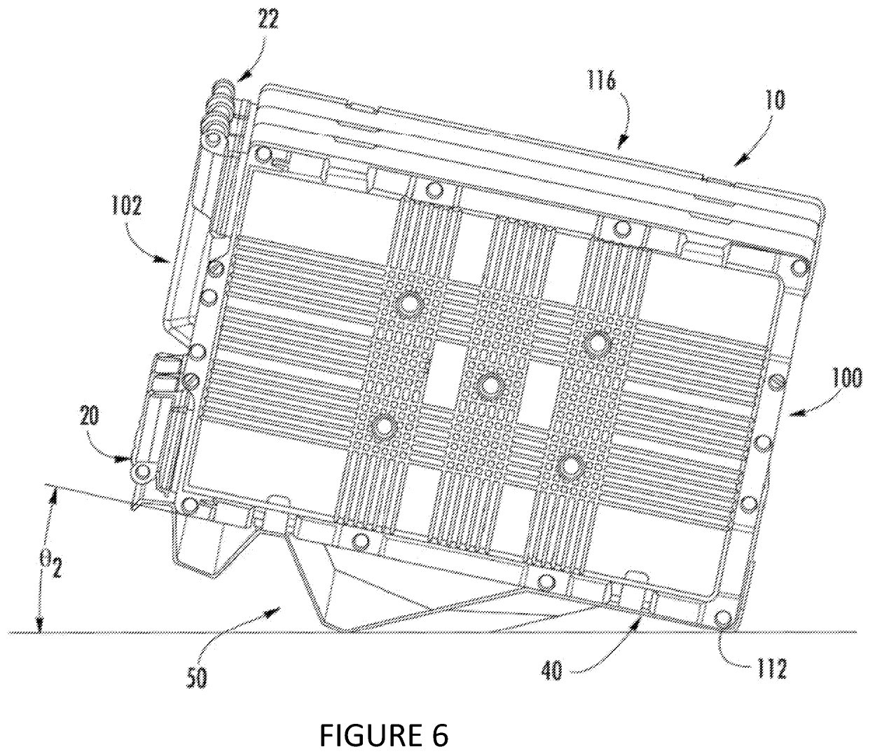

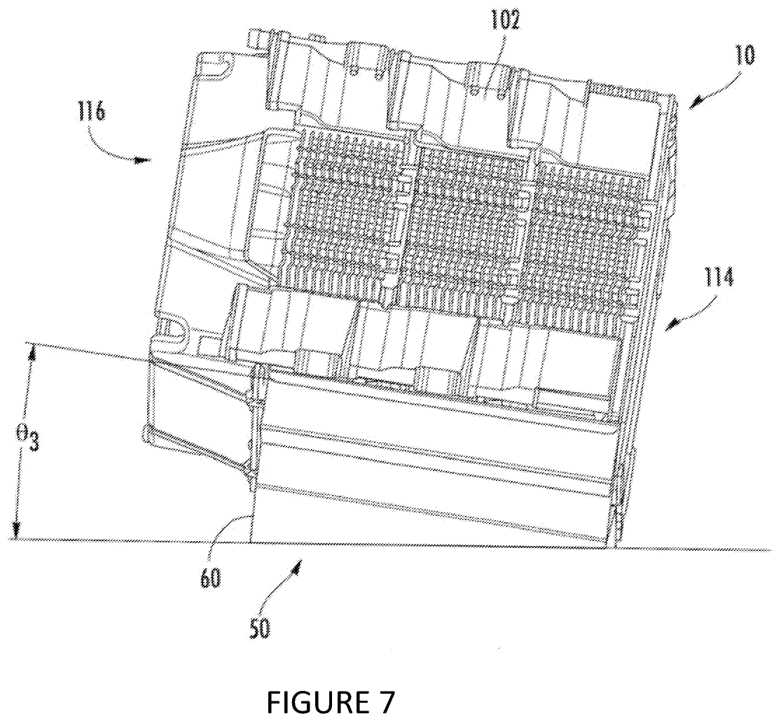

Referring to FIG. 6, the multi-position support 50 and the cell culture apparatus 10 are shown in the tilted configuration where the front 102 is now more elevated than the rear 100 providing an angle θ₂ (between 11 degrees and 13 degrees) to horizontal. As can be seen, a corner 112 between the side 40 and the rear 100 of the cell culture apparatus 10 rests against the support member in the tilted configuration. Referring to FIG. 7, a top 116 is more elevated than the bottom 114 at an angle θ₃ (between seven degrees and nine degrees) to horizontal. The tilted configuration thereby provides the multi-position support 50 and the cell culture apparatus 10 with the compound angle of both θ₂ (front to rear) and θ₃ (top to bottom), which may be referred to as an end fill position. Once the cell culture apparatus 10 is filled, the side 60 of the multi-position support 50 nearest the top 116 of the cell culture apparatus 10 may be rotated upward until the cell culture apparatus 10 is in an upright, standing position. Thus, the cell culture apparatus 10 can be manipulated throughout the fill process using only the multi-position support 50 without any need for lifting the cell culture apparatus 10 from the multi-position support 50. Emptying the cell culture apparatus 10 can be performed in a reverse order.

The above-described multi-position supports can be used to manipulate cell culture apparatuses without any need for handling the cell culture apparatuses separately from the multi-position supports during a fill or empty operation. The multi-position supports can thereby increase process efficiency and save users time due to higher fill and empty rates as well as from simple quick angle change procedures. The multi-position supports may further provide clear and concise control protocols which can reduce errors, reduce the possibility of product failure and/or damage, reduce angle variations due to method cradling using the multi-position supports and fixed tilt angles. Providing the multi-position supports with a compound tilt angle reduces the change of wetting out the filter attached to the air manifold. In some embodiments, the multi-position devices may be formed of stainless steel, which can provide increased durability and meet good manufacturing practices (GMP). The multi-position apparatuses may be formed from a sheet material on a metal brake to reduce manufacturing costs. Modifications can be made without incurring significant costs for re-tooling.

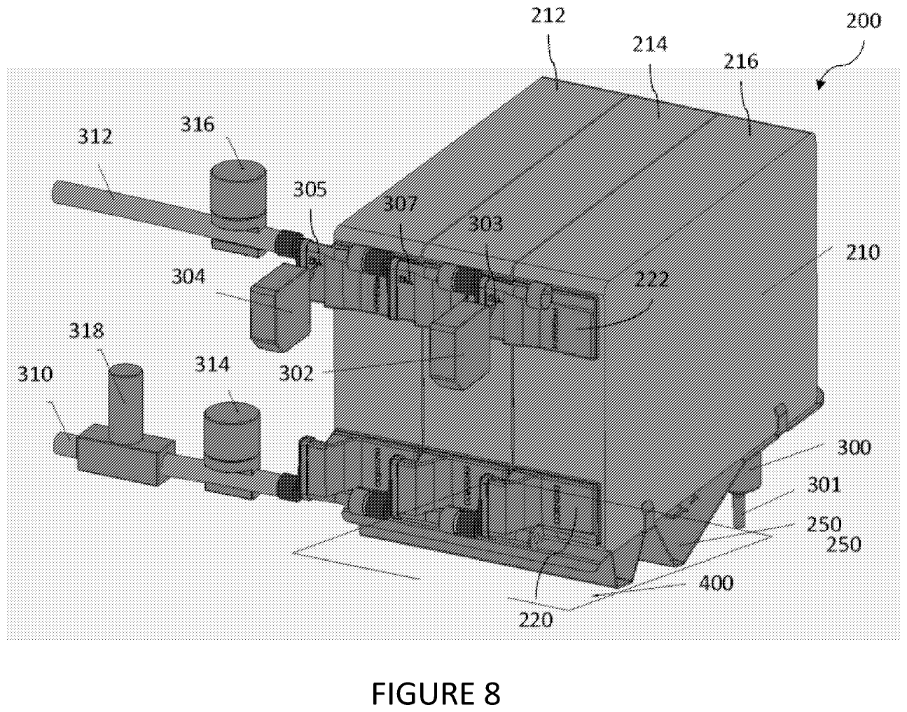

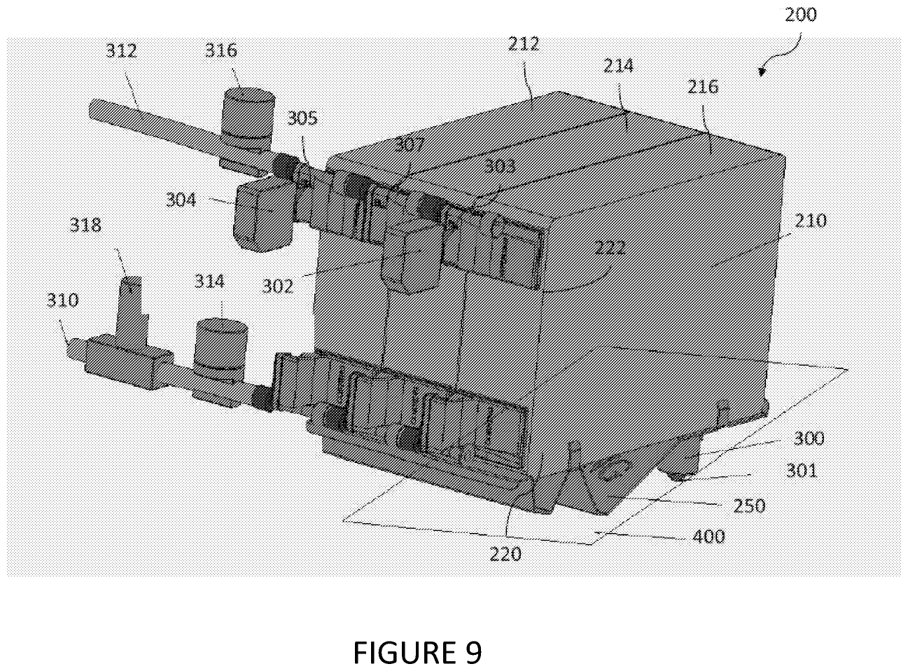

Referring to FIGS. 8 and 9, a cell culture system 200 is shown the enables automated filling of a cell culture apparatus 210. The cell culture apparatus 210 is not shown in detail in FIGS. 8 and 9, but it can have the same structure and features of the cell culture apparatus described elsewhere in this disclosure. Likewise, FIGS. 8 and 9 show a multi-position support 250 similar to the multi-position support described elsewhere in this disclosure. Accordingly, the following description will focus on the additional components and features shown in FIGS. 8 and 9 as they relate to an automated filling cell culture system 200.

The cell culture apparatus 210 includes three cell culture modules 212, 214, and 216, each containing multiple layers of cell culture chambers (see FIG. 2), and are stacked, one on top of the other, to form the multiple layer cell culture apparatus 210. Each cell culture module 212, 214, and 216 are equipped with two manifolds 220 and 222. Liquid may enter and exit the cell culture modules 212, 214, and 216 through the first manifold 220. Thus, the first manifold 220 may be referred to as a fluid manifold. Air may enter and exit the cell culture modules 212, 214, and 216 through the second manifold 222. Thus, the second manifold 222 may be referred to as an air manifold.

The system 200 includes a first fill sensor 302 arranged to detect the presence of liquid media within the cell culture apparatus 210 at a first fill level 303. A second fill sensor 304 can also be provided to detect the presence of liquid media within the cell culture apparatus 210 at a second fill level 305. Optionally, a third or more fill sensors (not shown) can be provided for additional functionality. For example, a third fill sensor could be provided to detect the presence of liquid media within the cell culture apparatus 210 at a third fill level 307. This third fill level 307 can be used, for example, to alter (e.g., slow) a rate of filling the cell culture apparatus 210 with liquid media. As shown in FIGS. 8 and 9, the location of the first, second, and third fill levels 303, 305, 307 are located on the second manifold 222. The location of the fill levels 303, 305, 307 on the manifold provides a useful and accessible location to measure the fill level of the cell culture apparatus 210, because these locations are at the top of the cell culture apparatus 210 during filling and thus represent fill levels that are progressively closer to a final fill level of the cell culture apparatus 210. In addition, the manifold 222 can provide a clear and unobstructed view of the liquid media level, allowing it to be easily detected by a variety of sensor types. In some embodiments, the second fill level 305 indicates a final fill level of the cell culture apparatus 210. The fill sensors 302, 304 can be any type of sensor known in the art capable of detecting liquid media reaching a certain position in the cell culture apparatus 210. In some embodiments, the sensors include at least one of an optical sensor, a through-beam sensor, or a photoelectric sensor.

The cell culture system 200 also includes an actuator 300 arranged to move the cell culture apparatus 210 from a first orientation (e.g., FIG. 8) to a second orientation (e.g., FIG. 9) during filling. The plane 400 in FIGS. 8 and 9 represents the plane of a support member (e.g., table, lab bench, or other generally horizontal surface on which the system 200 is supported). In some preferred embodiments, the cell culture apparatus 210 is moved by the actuator 300 from the first orientation shown in FIG. 8, where a piston 301 of the actuator 300 is extended, to the second orientation shown in FIG. 9, where a piston 301 of the actuator has been retracted, when the first fill sensor 302 detects that liquid media has reached the first fill level 303. In a further aspect of some embodiments, the actuator 300 can move the cell culture apparatus 210 to yet another orientation upon another fill sensor detecting liquid media at a given fill level. The actuator 300 can include a solenoid or other mechanism (e.g., piezoelectric, pneumatic or hydraulic piston, or motor) to extend (e.g., FIG. 8) and retract (e.g., FIG. 9) the actuator 300 as needed to achieve the desired orientation. The actuator 300 can be integral with or attachable to the multi-position support 250.

The fill sensors 302, 304 and the actuator 300 can be connected to a controller (not shown). In particular, the fill sensors 302, 304 can send detection signals to the controller upon detecting that the liquid media reaches a given fill level (e.g., 303, 305). The controller can then control the actuator 300 to change the orientation of the cell culture apparatus 210, or to control other components of the system (such as a pump or valves) to control the rate of filling by controlling the flow of liquid media into the cell culture apparatus 210. The fill sensors can also be coupled with timers so that the functions of the cell culture system 200 can be triggered on a timer delay initiated by a signal from the fill sensors.

In some embodiments, a detection signal by the first fill sensor 302 can cause the filling to be stopped temporarily until the cell culture apparatus is in the desired, second orientation, at which point filling can be resumed.

The cell culture system 200 can further include a filling port 310 (e.g., tubing or other fluid conduit) connected to an inlet of the first manifold 220. The filling port 310 can be connected to a pump or other means of advancing liquid into the cell culture apparatus 210. Connected to the second manifold 222 is a vent port 312 for venting fluid (e.g., air) from an outlet of the second manifold 222 during filling.

In addition, the cell culture system 200 can include various other valves and sensors to assist in monitoring and controlling the filling process. For example, one or more of an inlet valve 314 and an outlet valve 316 can be provided on or in the filling port 310 and vent port 312, respectively. The inlet valve 314 can close or restrict the filling port 310 to control the flow of liquid into the cell culture apparatus 210. The outlet valve 316 can close the vent port 312 so that air or other fluid cannot escape the cell culture apparatus, effectively stopping the filling process. In some embodiments, the inlet and outlet valves 314, 316 are pinch valves to pinch and close the inlet and outlet, respectively. For example, the inlet and outlet valves 314, 316 can be solenoid pinch valves. Because pinch valves can be employed outside of the tubing, they are easy to reuse. In addition, such pinch valves can be small enough to be out of the way of disposable clamps that may be used by an operator of the system, so that the operator would simply clamp off the lines with the disposable clamps before releasing the pinch valves.

In addition, various flow or pressure sensors can be used in the cell culture system 200. As shown in FIGS. 8 and 9, a flow control sensor 318 can be provided on the filling port 310. In some embodiments, the flow control sensor 318 is an inline pressure sensor to monitor the pressure of the liquid media during filling. The flow control sensor 318 can be connected to a controller controlling the pump to adjust the flow rate of the liquid media based on feedback from the flow control sensor 318. For example, if the pressure is too high, the pump speed can be lowered automatically. Such a pressure sensor can be equipped with a stainless foil face or other surface than can be cleaned after use, so that the sensor can be reused.

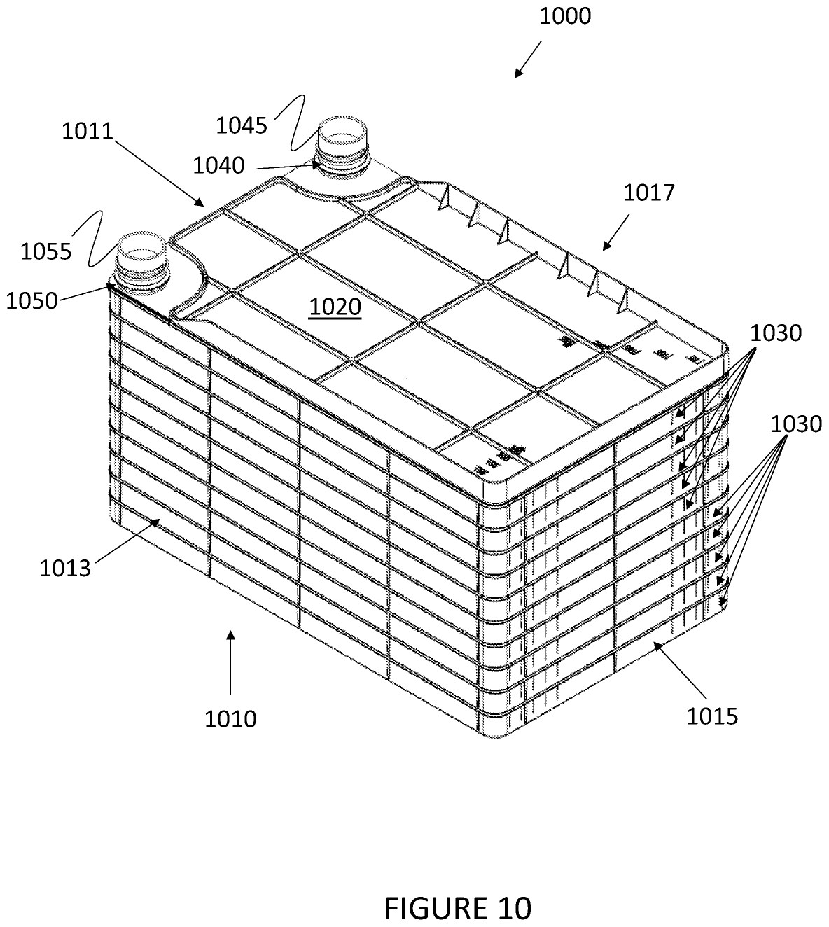

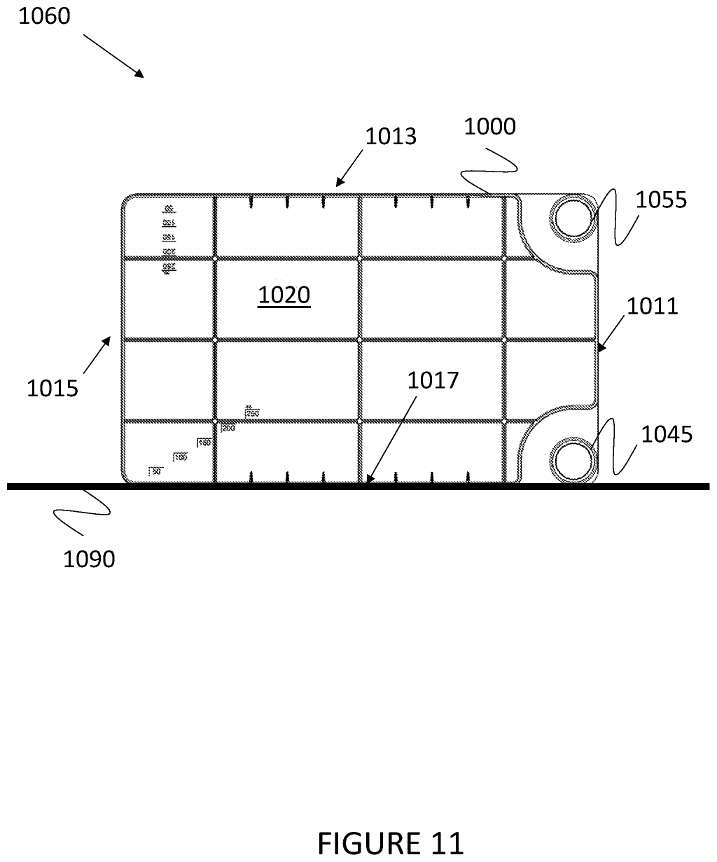

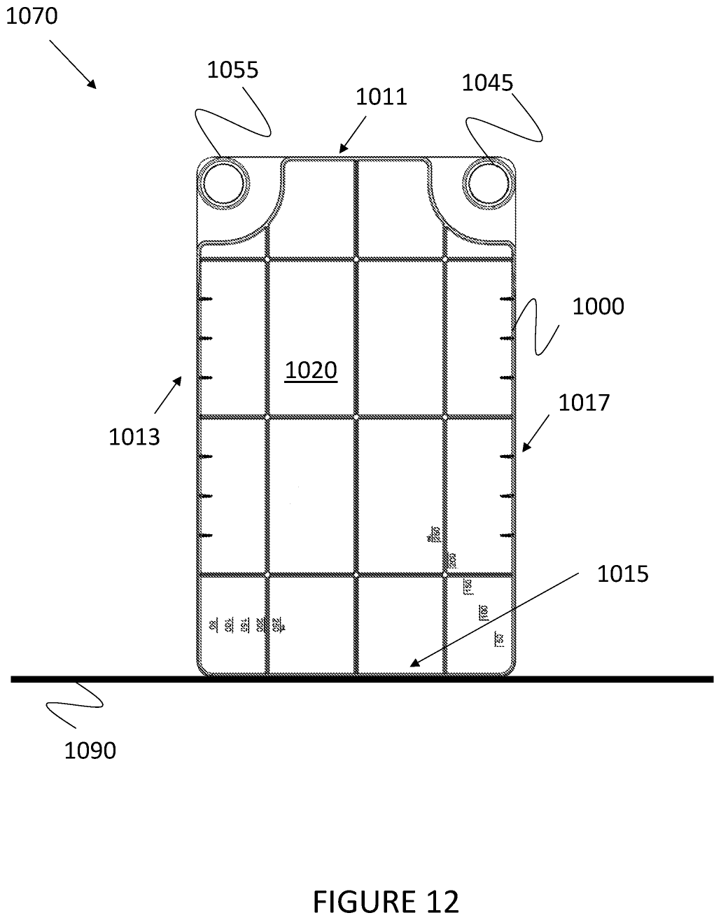

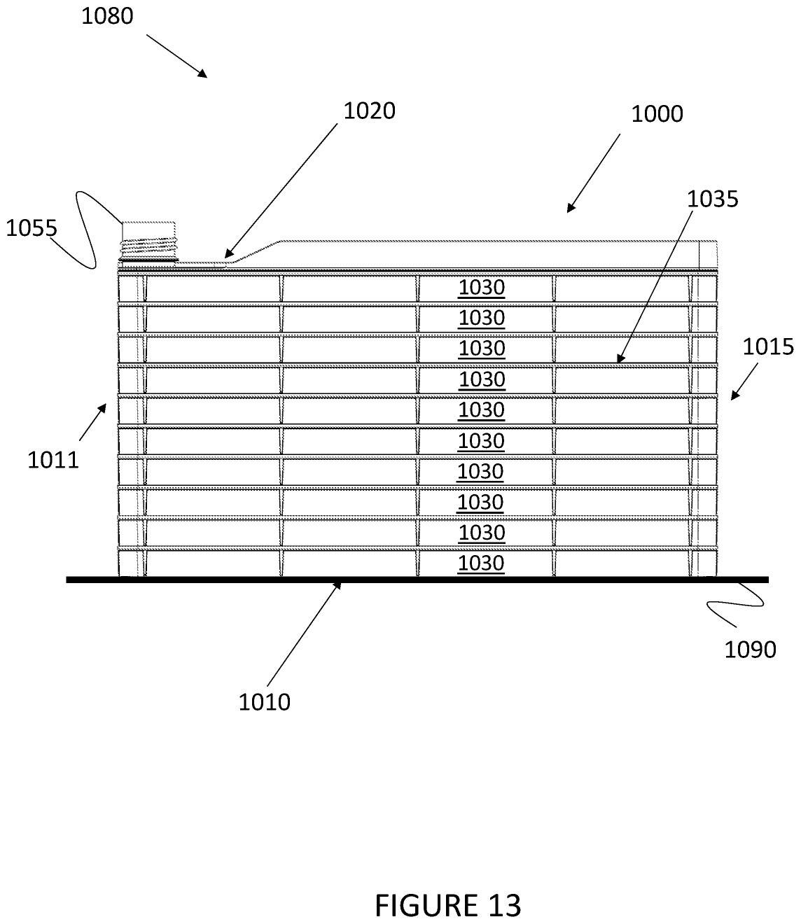

In some embodiments, systems, devices, and methods for autofill described herein may be used with a cell culture vessel that comprises multiple layers or stacks. For example, in some embodiments, the cell culture vessel may be a CellSTACK® culture vessel available from Corning Incorporated (Corning, N.Y.). Such a cell culture vessel 1000 is shown in FIGS. 10-13. FIG. 10 shows a perspective view of a cell culture apparatus or vessel 1000, according to one or more embodiments shown and described herein. FIG. 11 shows a side view of the cell culture apparatus 1000 of FIG. 10 in a first fill orientation 1060, according to one or more embodiments shown and described herein. FIG. 12 is a side view of the cell culture apparatus 1000 of FIG. 10 in a second fill orientation 1070, according to one or more embodiments shown and described herein. FIG. 13 is a side view of the cell culture apparatus 1000 of FIG. 10 in an incubation orientation 1080, according to one or more embodiments shown and described herein.

As shown in FIGS. 10-13, the cell culture vessel 1000, such as a CellSTACK® culture vessel, comprises a flat bottom surface 1010 and a flat top surface 1020 opposite the bottom surface 1010 and four side walls 1011, 1013, 1015, 1017 extending vertically between the bottom surface 1010 and the top surface 1020. A plurality of layers, cell culture chambers, or stacks 1030 may be disposed between the bottom surface 1010 and top surface 1020, each layer or cell culture chamber 1030 comprising a bottom culture surface 1035, wherein one side 1011 of each cell culture chamber 1030 is in communication with a fill column 1040 and a vent column 1050. The cell culture vessel may comprise any suitable number of cell culture chambers 1030. Nonlimiting examples include cell culture vessels comprising 1 stack or culture chamber, 2 stacks or culture chambers, 5 stacks or culture chambers, 10 stacks or culture chambers, or 40 stacks or culture chambers.

The fill column 1040 and fill port 1045 and the vent column 1050 and vent port 1055 allow direct access to the chamber bottom 1035, providing greater flexibility for sterile filling and emptying by pouring, pipetting or via tubing in a fully enclosed system. The fill column 1040 and vent column 1050 may have access ports 1045, 1055 at the top surface 1020 of the cell culture vessel 1000, wherein the access ports may be threaded to engage with a cap or tubing. In some embodiments, the fill port 1045 and vent port 1055 are configured to engage with a cap, such as by threading or other suitable connection. In some embodiments, a cap may comprise a porous and/or hydrophobic membrane to allow gas exchange while minimizing the risk of contamination. In some embodiments, caps may comprise filling caps available with integrally sealed tubing to allow direct sterile transfer of media and cells via pumping or gravity. The fill column 1040 and vent column 1050 may be located on the same side 1011 of the cell culture vessel 1000, at opposite ends of that side 1011. The fill column 1040 extends vertically from the bottom surface 1010 of the cell culture vessel to the top surface 1020 of the cell culture vessel and is in communication with each layer or culture chamber 1030 therein, wherein the fill column 1040 comprises a fill port 1045 at the top surface 1020 of the cell culture vessel 1000, and wherein a desired cap and/or desired tubing may be attached to the fill port 1045. The vent column 1050 extends vertically from the bottom surface 1010 of the cell culture vessel to the top surface 1020 of the cell culture vessel and is in communication with each layer or culture chamber 1030 therein, wherein the vent column 1050 comprises a vent port 1055 at the top surface 1020 of the cell culture vessel 1000, and wherein a desired cap and/or desired tubing may be attached to the vent port 1055.

In some embodiments, the chambers 1030 of the cell culture vessel 1000 may be filled by an autofill system as described in embodiments disclosed herein. In other embodiments, the chambers of the cell culture vessel may be filled by any suitable means, such as by gravity, by peristaltic pump, or by pouring. In some embodiments, the cell culture vessel 1000 may be attached to autofill systems and components disclosed herein, such as systems that may include fill sensors, to automatically fill the cell culture chambers 1030 of the cell culture vessel 1000 with minimal user interference. Aseptic connections may be made in a laminar flow hood or clean room environment. Cell culture vessels may be moved to a nonsterile environment if a closed filling system is used, such as a filling system by gravity or pumping.

When filling the cell culture vessel 1000, a cap, such as a standard vent cap, on the filling port 1045 may be replaced by a filling cap. The cell culture vessel 1000 may be moved to a first fill orientation 1060 wherein the cell culture vessel 1000 may be placed on its side 1011 with the filling port 1045 near the bottom-most part of the vessel where the vessel is resting or supported on a work surface 1090. In some embodiments, a standard vented cap on the venting port 1055 may be replaced to a vented filling cap with air venting filter to reduce back pressure. The filling cap tubing may be connected to tubing from a sterile dispensing vessel containing a cell suspension. In some embodiments, a cell suspension from a dispensing vessel may be pumped into the cell culture vessel to fill the cell culture chambers. The chambers may initially fill unevenly, but the medium will level out in each chamber or stack of the cell culture vessel.

Once filling is complete, the cell culture vessel may be moved to a second fill orientation 1070. In the first fill orientation 1060, the cell culture vessel is placed on its side 1011 with the filling port 1045 near the bottom. In the second fill orientation 1070, the cell culture vessel is moved 90 degrees so that the side 1011 comprising the filling port 1045 and venting ports 1055 are located at a topmost position of the vessel. In the second fill orientation 1070, side 1015, which is opposite side 1011, is resting on or supported by the support or work surface 1090. The filling cap on the filing port 1045 may be replaced, such as with a vented or solid cap.

The cell culture vessel may then be placed in an incubation position 1080, where the bottom surface 1010 and top surface 1020 of the cell culture vessel 1000 and the bottom culture surface 1035 of the culture chambers 1030 are in a horizontal position. For example, the cell culture vessel 1000 may have the top surface 1020 and bottom surface 1010 in a flat, horizontal position parallel to a work surface 1090 upon which the cell culture vessel rests. Thus, when the cell culture vessel 1000 is moved to the incubation position 1080, gravity allows for the cell culture medium to cover the surface of each chamber or stack within the cell culture vessel. In some embodiments, a user or manipulator may carefully tilt the cell culture vessel 1000 to assist with covering the surface of each cell culture chamber with medium but ensuring that the medium does not flow over the edge of the chambers into the access column. In some embodiments, a user manually manipulates or positions the cell culture chamber into the various positions, such as the first fill orientation 1060, the second fill orientation 1070, and the incubation orientation 1080. In some embodiments, the steps of positioning the cell culture vessel for filling are automated and conducted by a manipulator. In embodiments, the manipulator is sized and configured to accommodate one or more cell culture vessels and may manipulate or position the cell culture vessels for filling automatically or with minimal manual involvement by the user. In some embodiments, the manipulator may be a manipulator commercially available from Shikoku Industries Co. Ltd. (Tokushima, Japan).

The cell culture vessel may then be placed in an incubator or warm room on a level, flat surface that supports the weight of the cell culture vessel. After suitable incubation, the cell culture vessel may be emptied or may require a media exchange. To empty the cell culture vessel, the filling port cap may be replaced with a filling cap. The cell culture vessel may be connected to a sterile collection vessel, and the medium from the cell culture vessel may be transferred to the collection vessel, such as by gravity or pumping.

Thus, embodiments of cell culture autofill systems and related methods are disclosed. One skilled in the art will appreciate that the cell culture autofill systems and methods described herein can be practiced with embodiments other than those disclosed. The disclosed embodiments are presented for purposes of illustration and not limitation.

What is claimed is:

1. A cell culture system comprising:
a cell culture vessel comprising a cell culture chamber for culturing cells, an inlet through which liquid can flow for filling the cell culture vessel, and an outlet through which fluid can exit the cell culture vessel;
at least one fill sensor arranged to detect liquid within the cell culture vessel reaching a fill level at a predetermined position of the cell culture vessel during filling of the cell culture vessel, the at least one fill sensor configured to generate a detection signal when liquid within the cell culture vessel reaches the fill level; and
an actuator configured to change an orientation of the cell culture vessel in response to the detection signal.

2. The cell culture system of claim 1, wherein the at least one fill sensor comprises a first fill sensor arranged to detect liquid within the cell culture vessel reaching a first fill level and configured to generate a first detection signal when liquid within the cell culture vessel reaches the first fill level,
wherein the actuator is configured to change the orientation of the cell culture vessel from a first orientation to a second orientation in response to the first detection signal.

3. The cell culture system of claim 2, wherein the at least one fill sensor comprises a second fill sensor arranged to detect liquid within the cell culture vessel reaching a second fill level and configured to generate a second detection signal when liquid within the cell culture vessel reaches the second fill level,
wherein the system is configured to stop filling the cell culture vessel in response to the second detection signal, and
wherein the second fill level is different than the first fill level.

4. The cell culture system of any one of claims 1, wherein the cell culture vessel is a multi-layered cell culture vessel.

5. The cell culture system of claim 1, the system further comprising a controller configured to receive the detection signal of the at least one sensor and to control the actuator in response to the detection signal.

6. The cell culture system of claim 1, further comprising a pump fluidly connected to the inlet of the cell culture vessel.

7. The cell culture system of claim 6, wherein the controller is configured to control a flow rate of the pump.

8. The cell culture system claim 2,
wherein the cell culture comprises a fill side and a support-facing side, the fill side being where the inlet and the outlet are disposed, and the support-facing side being adjacent to the fill side and facing a substantially horizontal support member that is underneath the cell culture vessel in the first orientation,
wherein, in the first orientation, the support-facing side is at a first angle relative to the support member, and the outlet is at a first distance from the support member, the inlet being closer to the support member than the outlet, and
wherein, in the second orientation, the support-facing side is at a compound angle relative to the support member, the compound angle comprising (i) a second angle between a length of the support-facing side and the support member and (ii) a third angle between a width of the support-facing side and the support member, and
wherein, in the second orientation, the outlet is at a second distance from the support member, the second distance being greater than the first distance.

9. The cell culture system of claim 1, wherein the at least one fill sensor is attached to an exterior of the cell culture vessel and detects the fill level through a wall of the cell culture vessel.

10. The cell culture system of claim 9, wherein the cell culture vessel comprising multiple cell culture chambers and a manifold connecting the multiple cell culture chambers, the at least one fill sensor being attached to the manifold.

11. The cell culture system of claim 1, wherein the at least one fill sensor is detachable from the cell culture vessel and is reusable.

12. The cell culture system of claim 1, further comprising a flow control arranged to detect in-line fluid pressure of the system,
wherein the flow control is configured to reduce a flow rate of the system in response to the in-line fluid pressure being equal to or greater than a predetermined pressure value.

13. The cell culture system of claim 1, further comprising a valve at an outlet of the cell culture vessel,
wherein the valve is configured to close a fluid path at or near the outlet of the cell culture vessel to stop filling of the cell culture vessel.

14. The cell culture system of claim 13, wherein the valve is a pinch valve on an exterior of tubing connected to the outlet.

15. The cell culture system of claim 13, wherein the valve is configured to close the fluid path in response to at least one of the detection signal from one of the at least one fill sensors and an in-line fluid pressure.

16. The cell culture system of claim 1, wherein the at least one fill sensor comprises at least one of an optical sensor, a through-beam sensor, or a photoelectric sensor.

17. The cell culture system of claim 1, further comprising a multi-position support configured to support the cell culture vessel.

18. The cell culture system of 17, wherein the actuator is attached to the multi-position support and is configured to change an orientation of the multi-position support relative to a horizontal support surface upon which the multi-position support is disposed.

19. The cell culture system of claim 17, wherein the actuator comprises an adjustable length comprising a first length and a second length, and
wherein at the first length the cell culture vessel is in the first orientation and at the second length the cell culture vessel is in the second orientation.

20. The cell culture system of claim 19, wherein the first length is greater than the second length.

21. The cell culture system of claim 1, wherein the actuator comprises at least one of a solenoid, a piezoelectric material, a pneumatic piston, and a hydraulic piston.

22. The cell culture system of claim 3, further comprising a third fill sensor arranged to detect liquid within the cell culture reaching a third fill level and configured to generate a third detection signal when liquid within the cell culture vessel reaches the third fill level, the third fill level being different than the first fill level and the second fill level.

23. The cell culture system of claim 22, wherein the controller is configured to slow a filling rate of the cell culture vessel based on a third detection signal from the third fill sensor.

24. The cell culture system of claim 3, wherein, during filling of the cell culture vessel with a liquid, the liquid reaches the first fill level before the second fill level.

25. The cell culture system of claim 24, wherein, during filling of the cell culture vessel with a liquid, the liquid reaches the third fill level after the first fill level and before the second level.

26. The cell culture system of claim 25, wherein the controller is configured to stop filling the cell culture vessel based on the second detection signal from the second fill sensor.

27. The cell culture system of claim 17, wherein the multi-position support comprises:
a primary base that rests against the support member in an upright configuration;
a support surface that is offset vertically from the primary base in the upright configuration, the support surface supporting the cell culture vessel with the cell culture vessel located thereon; and
an intermediate surface that extends between the primary base and the support surface, wherein the intermediate surface meets the primary base at an interface that extends at an oblique angle to sides of the primary base;
wherein the multi-position support has a tilted configuration where the multi-position support is rotated about the interface such that the support surface is closer to the support member than in the upright configuration with the support surface supporting the cell culture vessel thereon.

28. The cell culture system of claim 27, wherein the multi-position support further comprises a secondary base that rests against the support member in the upright configuration.

29. The cell culture system of claim 28, wherein the support surface is a first support surface, the multi-position support further comprising a second support surface located between the primary base and the secondary base, the second support surface supporting the cell culture vessel with the cell culture vessel located thereon.

30. The cell culture system of claim 29, wherein the first support surface and the second support surface lie in substantially a same plane that is oblique to the primary base.

31. The cell culture system of claim 30, wherein the multi-position support further comprises a support flange that engages a fill side of the cell culture vessel to constrain the cell culture vessel on the first and second support surfaces.

32. The cell culture system of claims 29, wherein the multi-position support further comprises another support flange that extends outward from the first support surface that engages a rear side of the cell culture vessel that is opposite the fill side.

33. The cell culture system of claim 29, wherein the second support surface is spaced from the support member.

34. The cell culture system of claim 27, wherein the support surface meets the intermediate surface at another interface that is at an oblique angle to sides of the primary base.

35. The cell culture system of claim 34, wherein the oblique angles of both interfaces to the sides of the primary base are about the same.

36. A method of changing a fill angle of a cell culture apparatus comprising cell culture vessel comprising multiple cell culture modules, the multiple cell culture modules being fluidly connected together by a fluid manifold and an air manifold, the method comprising:
connecting the cell culture apparatus to a multi-position support, the multi-position support comprising a tilted configuration and an upright configuration;
filling the cell culture apparatus while supported by the multi-position support with the multi-position support in either the upright configuration or the tilted configuration;
detecting with a first fill sensor exterior to the cell culture modules when the cell culture apparatus has been filled to a first fill level;
changing the multi-position support from one of the tilted configuration and the upright configuration to the other of the tilted configuration and the upright configuration, based on the detecting by the first fill sensor;
detecting with a second fill sensor exterior to the cell culture modules when the cell culture apparatus has been filled to a second fill level different from the first fill level; and
stopping the filling based on the detecting by the second fill sensor.

37. The method of claim 36, wherein the multiple cell culture modules each contain multiple layers of cell culture chambers.

38. The method of claim 36, wherein the multi-position support comprises:
a primary base that rests against a support member in the upright configuration; a support surface that is offset vertically from the primary base in the upright configuration, the support surface supporting the multi-layer cell culture apparatus with the multi-layer cell culture apparatus located thereon; and
an intermediate surface that extends between the primary base and the support surface, wherein the intermediate surface meets the primary base at an interface that extends at an oblique angle to sides of the primary base;
wherein, in the tilted configuration, the multi-position support is rotated about the interface such that the support surface is closer to the support member than in the upright configuration with the support surface supporting the multi-layer cell culture apparatus thereon.

39. The method of claim 36, wherein the step of filling comprises filling the cell culture apparatus while supported by the multi-position support with the multi-position support in the upright configuration.

40. The method of claim 37, further comprising tilting the cell culture apparatus using the multi-position support by rotating the multi-position support about the interface.

41. The method of claim 36, wherein the changing of the multi-position support from one of the tilted configuration and the upright configuration to the other comprises moving an actuator attached to the multi-position support.

42. The cell culture system of claim 5,
wherein the cell culture comprises a fill side and a support-facing side, the fill side being where the inlet and the outlet are disposed, and the support-facing side being adjacent to the fill side and facing a substantially horizontal support member that is underneath the cell culture vessel in the first orientation,
wherein, in the first orientation, the support-facing side is at a first angle relative to the support member, and the outlet is at a first distance from the support member, the inlet being closer to the support member than the outlet, and
wherein, in the second orientation, the support-facing side is at a compound angle relative to the support member, the compound angle comprising (i) a second angle between a length of the support-facing side and the support member and (ii) a third angle between a width of the support-facing side and the support member, and
wherein, in the second orientation, the outlet is at a second distance from the support member, the second distance being greater than the first distance.

43. The cell culture system of claim 8, further comprising a multi-position support configured to support the cell culture vessel.

44. The cell culture system of claim 43, further comprising a third fill sensor arranged to detect liquid within the cell culture reaching a third fill level and configured to generate a third detection signal when liquid within the cell culture vessel reaches the third fill level, the third fill level being different than the first fill level and the second fill level.

45. The cell culture system of claim 27,
wherein the cell culture comprises a fill side and a support-facing side, the fill side being where the inlet and the outlet are disposed, and the support-facing side being adjacent to the fill side and facing a substantially horizontal support member that is underneath the cell culture vessel in the first orientation,
wherein, in the first orientation, the support-facing side is at a first angle relative to the support member, and the outlet is at a first distance from the support member, the inlet being closer to the support member than the outlet, and
wherein, in the second orientation, the support-facing side is at a compound angle relative to the support member, the compound angle comprising (i) a second angle between a length of the support-facing side and the support member and (ii) a third angle between a width of the support-facing side and the support member, and
wherein, in the second orientation, the outlet is at a second distance from the support member, the second distance being greater than the first distance.

46. The cell culture system of claim 27, further comprising a third fill sensor arranged to detect liquid within the cell culture reaching a third fill level and configured to generate a third detection signal when liquid within the cell culture vessel reaches the third fill level, the third fill level being different than the first fill level and the second fill level.
